(19) United States
(12) Patent Application Publication
(10) Pub. No.: US 2016/0369814 A1
(43) Pub. Date: Dec. 22, 2016

(54) PUMP HOUSING WITH HARD INNER LAYER AND WELDABLE OUTER LAYER

(71) Applicant: HERAEUS DEUTSCHLAND GMBH & CO. KG, Hanau (DE)

(72) Inventors: Stefan SCHIBLI, Frankfurt (DE); Jens TRÖTZSCHEL, Ronneburg (DE)

(73) Assignee: HERAEUS DEUTSCHLAND GMBH & CO. KG, Hanau (DE)

(21) Appl. No.: 14/900,513

(22) PCT Filed: Jun. 20, 2014
(86) PCT No.: PCT/EP2014/001685

§ 371 Date: Dec. 21, 2015

(30) Foreign Application Priority Data
Jun. 21, 2013 (DE) 10 2013 211 845.7

(51) Int. Cl. F04D 29/52; F04D 13/02; A61M 1/10; F04D 29/02

(52) U.S. Cl. CPC F04D 29/528; F04D 29/026; F04D 13/024; A61M 1/1012; F05B 2240/30; F05B 2240/142; F05B 2280/20041; F05B 2280/20043; F05B 2280/103; F05B 2280/1043; F05B 2280/107

(57) ABSTRACT

One aspect is a pump device comprising an impeller, and a pump housing which at least partly surrounds an interior region and has an inlet and an outlet, wherein the impeller is provided in the interior region of the pump housing. The pump housing comprises a composite of a first hollow body and a further hollow body which at least partly surrounds the first hollow body on the side facing away from the interior region. At least 60% of the surface area of the side of the first hollow body facing away from the interior space is connected to the further hollow body. The further surface of the first hollow body facing the interior region of the pump housing has a hardness of at least 330 HV.

### BACKGROUND

One aspect of the invention relates to a pump device comprising i. an impeller; ii. a pump housing which at least partly surrounds an interior region and has an inlet and an outlet, wherein the impeller is provided in the interior region of the pump housing; wherein the pump housing comprises a composite of a first hollow body and a further hollow body which at least partly surrounds the first hollow body on the side facing away from the interior region; wherein at least 60% of the surface area of the side of the first hollow body facing away from the interior space is connected to the further hollow body, based on the surface area of the side of the first hollow body facing away from the interior space, wherein the pump housing faces the interior region with a further surface of the first hollow body; wherein the further surface of the first hollow body facing the interior region of the pump housing has a hardness of at least 330 HV, wherein the further hollow body has a metal content of at least 50% by weight, based on the total mass of the further hollow body. One aspect of the invention further relates to a housing having the features described above for the pump housing.

In addition, one embodiment of the invention relates to a process for producing a pump housing, which comprises the steps: a. provision of a first hollow body; b. provision of a further hollow body, wherein at least the first hollow body is configured as a cylindrical hollow body; c. contacting of the first hollow body with the further hollow body to form a composite, wherein the contacting is performed as force-fitting or material-fitting connection of the first hollow body and the second hollow body.

Pump devices having rotors or impellers are known. Some pump devices have a pump housing in the form of a tube as transport section for a fluid to be conveyed. An impeller which, for example, is driven by a motor located outside the transport section by means of a drive shaft is frequently located therein. The pump housing is fastened by means of one or more holding elements to the pump device. This type of attachment can have various disadvantages. Firstly, an additional working step is required for attaching the holder. This increases production costs and is inefficient in terms of resources. Furthermore, the connection between the pump housing and the holder is not without tension as a result of the method of production or because of the connecting means used, e.g. screws or rivets. This is due to the fact that the materials selected for the holders and/or connecting means are usually different from those selected for the pump housing. Due to these tensions, the connections of the holder to the pump housing deteriorate over time. In addition, it is extremely important for space to be saved, especially for very small pumps. This applies particularly to pumps which are to be implanted in a body. This is more difficult to realize in the case of pumps having a plurality of individual parts than in the case of a pump having a smaller number of individual parts.

In general, it is an object of the present invention to at least partly overcome the disadvantages of the prior art.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Further measures and advantages of the invention are evident from the claims, the description provided hereinafter, and the drawings. The invention is illustrated through several exemplary embodiments in the drawings. In this context, equal or functionally equal or functionally corresponding elements are identified through the same reference numbers. The invention shall not be limited to the exemplary embodiments.

FIG. 1 schematically illustrates a pump device according to one embodiment of the invention.

FIG. 2 illustrates a flow diagram of a process for producing a pump housing according to one embodiment of the invention.

FIGS. 3a-e schematically illustrate, in different ways, a pump housing according to one embodiment of the invention having a first hollow body and a further hollow body arranged directly adjacent to one another.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

It is to be understood that the features of the various exemplary embodiments described herein may be combined with each other, unless specifically noted otherwise.

A further object is to provide a pump device whose materials are as biocompatible as possible, as easily processible as possible, as corrosion resistant as possible and can be durably connected to one another.

A further object is to provide a pump device which is designed to be as space-saving as possible.

Furthermore, it is an object of one embodiment of the invention to provide pump device being as tension-free as possible, in particular having a housing or pump housing being as tension-free as possible, and in particular to provide a passage from the pump housing to the remaining part of the pump device being as tension-free as possible.

An additional object is to provide a pump device which has, during use, an abrasion of the movable parts and the mountings thereof as low as possible.

In addition, it is an object of one embodiment of the invention to provide a pump housing for a pump device which can be integrated in an as simply and as space-saving manner as possible into other components, e.g. a component housing of the pump device.

In addition, it is an object of one embodiment of the invention to provide a pump housing for a pump device which can be connected to a component housing of the pump device in a hermetically sealed manner.

Furthermore, it is an object of one embodiment of the invention to provide a housing or pump housing which is as free as possible of internal and/or external tensions.

Furthermore, it is an object of one embodiment of the invention to provide a process by means of which a pump housing can be produced in a manner being as cost-saving and as time-saving as possible.

It is also an object of one embodiment of the invention to provide a component housing which is designed to be as space-saving as possible.

A further object is to provide a housing which can be joined in a hermetically sealed manner to other components.

A first object of one embodiment of the present invention is a pump device (10) comprising:

i. an impeller;

ii. a pump housing which at least partly surrounds an interior region and has an inlet and an outlet,

wherein the impeller is provided in the interior region of the pump housing;

wherein the pump housing comprises a composite of a first hollow body and a further hollow body which at least partly surrounds the first hollow body on the side facing away from the interior region;

wherein at least 60% of the surface area of the side of the first hollow body facing away from the interior space is connected to the further hollow body, based on the surface area of the side of the first hollow body facing away from the interior space,

wherein the pump housing faces the interior region with a further surface of the first hollow body;

wherein the further surface of the first hollow body facing the interior region of the pump housing has a hardness of at least 330 HV,

wherein the further hollow body has a metal content of at least 50% by weight, based on the total mass of the further hollow body.

The pump device of one embodiment of the invention is preferably suitable for being introduced into the body of a human being or an animal. The pump device of one embodiment of the invention is also preferably designed for conveying body fluids such as blood, serum, plasma, interstitial liquid, saliva or urine. In particular, the pump device of one embodiment of the invention is preferably introduced into the blood stream of a human being or animal in order to pump blood. The introduction of the pump device of one embodiment of the invention can, for example, comprise implantation into the body, placing on the body or connection to the body.

The pump housing of the pump device of one embodiment of the invention can have any shape which a person skilled in the art would select for use in a pump device. The pump housing preferably has at least one wall of the pump housing, hereinafter also referred to as pump housing wall. The at least one wall of the pump housing surrounds the interior region of the pump housing. The pump housing has at least two ends, with at least one inlet being arranged at one end and at least one outlet being arranged at the other end. The interior region of the pump housing is, apart from the inlet and outlet of the pump housing, completely surrounded by the wall. The pump housing can partly extend beyond the interior region of the pump housing. The pump housing preferably ends at the inlet or outlet.

The side of the pump housing facing away from the interior region will be referred to as the exterior of the pump housing. The pump housing preferably has an elongated shape. The shape of the pump housing is defined by a longitudinal extension and at least one cross section. A cross section of the pump housing is always determined in a plane perpendicular to the pump housing wall. If the pump housing wall is curved in its longitudinal extension, a cross section is determined perpendicular to the tangent at a point on the pump housing wall. The longitudinal extension is considered to be the extension of the pump housing in the pumping direction. The shortest, imaginary connecting line between the inlet and outlet within the pump housing is always applicable. The pump housing wall, also referred to as wall, extends in the direction of the longitudinal extension of the pump housing. The at least one wall can have one or more wall areas. If the pump housing has more than one wall area, these are connected via corners at which the wall areas come together. The wall, and preferably also the wall areas, of the pump housing preferably run parallel to the longitudinal extension of the pump housing. Part of the pump housing wall can extend beyond the interior region of the pump housing. The pump housing wall preferably extends over the entire interior region of the pump housing. If the pump housing has a tubular shape, the inlet is located at the first end and the outlet is located at the opposite end of the pump housing. At least part of the pump housing wall preferably ends at the ends of the pump housing. The part of the pump housing which protrudes beyond the interior region into the surroundings is referred to as pump housing tongue. In a preferred embodiment of the pump device of one embodiment of the invention, the pump housing has a first opening into the interior region at the first end, i. e. the inlet, and a further opening into the interior region at the further end, i. e. the outlet. The pump housing is fluidically connected to its surroundings via inlet and outlet. The openings at the ends of the pump housing make it possible for a fluid to flow through the interior region of the pump housing. The fluid is, for example, a gas, a liquid such as blood or a mixture thereof. The first opening preferably serves as point of introduction of the fluid to be conveyed in the interior region of the pump housing and the further opening serves as point of discharge of the fluid to be conveyed. The pump housing can have further openings, for example in the wall of the pump housing. These further openings can serve for the additional introduction of fluid or, on the other side, for the branched discharge of fluid. If the pump device of the invention is implanted in a body, for example in order to assist the flow of blood and thus take load off the heart, the pump device of one embodiment of the invention is connected via conduits to blood vessels of the body.

The pump housing comprises at least one cross section which is preferably selected from the group consisting of circular, rectangular or polygonal or ellipsoidal. The pump housing preferably has a longitudinal shape at least in one first section. Furthermore, the pump housing can comprise at least one further section whose shape is different from that of the first section of the pump housing.

The total length of the pump housing is preferably from 1.5 to 10 times, preferably from 2 to 9 times, or preferably from 2.5 to 8.5 times, longer than the internal diameter of the pump housing. The length of the pump housing is preferably determined along the outer wall of the pump housing in the pumping direction. The pump housing preferably has a length in the range from 1 mm to 10 cm, or preferably in the range from 2 mm to 8 cm, or preferably in the range from 5 mm to 5 cm. The pump housing preferably has an internal diameter in the range from 0.1 to 50 mm, or preferably in the range from 0.5 to 30 mm, or preferably in the range from 1 to 20 mm.

The wall, in particular the at least one wall area of the pump housing, is preferably smooth. Smooth means that the wall of the pump housing has a roughness in the range from 0.025 to 4 Ra, or preferably in the range from 0.05 to 3 Ra, or preferably in the range from 0.07 to 1 Ra.

The pump housing comprises at least one first hollow body and at least one further hollow body. The first hollow body and the further hollow body preferably differ in terms of their composition. The at least one first hollow body preferably has at least one, particularly preferably all, of the following properties:
a heat resistance as high as possible;
a pressure resistance as high as possible;
a hardness as high as possible;
a resistance to acids and base as high as possible s;
a roughness as low as possible;
a connectability to a metal or a metal-ceramic mixture (cermet) as tension-free as possible;
a sinterability with a metal or a metal-ceramic mixture (cermet) as good as possible;
an electrical conductivity as low as possible
a magnetic permeability as low as possible.

The at least one further hollow body preferably has at least one, preferably all, of the following properties:
a heat resistance as high as possible;
a pressure resistance as high as possible;
a hardness as high as possible;
a resistance to acids and bases as high as possible;
a roughness as low as possible;
a connectability to a metal as good as possible;
a weldability to a metal as good as possible.
a coefficient of thermal expansion as high as possible.

If the two first and second hollow bodies are assembled according to one embodiment of the invention in the production of the pump housing, it is possible to obtain a pump housing which combines one or more of the properties listed for the at least one first hollow body and the at least one further hollow body. At least part of the at least one first hollow body is connected to at least part of the further hollow body. The connection can be a direct connection or an indirect connection of the two hollow bodies. If the at least one first hollow body and the at least one further hollow body are directly connected to one another, the first hollow body and the further hollow body are connected to one another in a material-fitting or force-fitting manner.

A material-fitting connection is present in the case of direct connection when the materials properties of the first hollow body go over smoothly into the materials properties of the further hollow body. There is usually no sharp boundary between the two hollow bodies. Rather, there is usually a transition region in which the properties of the two hollow bodies mix. This transition region can also be referred to as intermediate region. In this intermediate region, the materials of the first hollow body have partially diffused into the materials of the further hollow body and preferably form a blended layer of the materials. The materials of the two hollow bodies are preferably bonded on an atomic or molecular level. Forces on an atomic or molecular level act on the materials of the first and further hollow bodies. A material-fitting connection can generally be released only by destruction of the materials. As an example of such a material-fitting connection, mention may be made of treatment of the adjoined hollow bodies at a temperature of preferably more than 500° C., or preferably more than 1000° C., or preferably more than 1500° C.

A further direct connection of the two hollow bodies in which a force-fitting connection is present between the hollow bodies is press fitting. This will be explained in more detail later.

An alternative force-fitting connection of the two hollow bodies is, for example, adhesive bonding of the two hollow bodies at the places of contact. Adhesive bonding will also be explained in more detail later.

The at least one first hollow body preferably comprises at least 40% by weight, preferably at least 70% by weight, or preferably at least 90% by weight, based on the total mass of the first hollow body, of a ceramic. The at least one first hollow body preferably comprises the ceramic in a proportion of from 40 to 100% by weight, or preferably in a proportion of from 70 to 100% by weight, or preferably in a proportion of from 80 to 100% by weight, based on the total mass of the first hollow body. The at least one first hollow body more preferably comprises 100% by weight, based on the total mass of the first hollow body, of the ceramic. The at least one first hollow body can comprise further materials. The further materials can be selected from the group consisting of water, an additive, a cermet or a mixture of at least two thereof.

The ceramic can be any ceramic which a person skilled in the art would select for the pump device of one embodiment of the invention. The ceramic is preferably selected from the group consisting of an oxide ceramic, a silicate ceramic, a nonoxidic ceramic and a mixture of at least two thereof.

The oxide ceramic is preferably selected from the group consisting of a metal oxide, a semimetal oxide and a mixture thereof. The metal of the metal oxide can be selected from the group consisting of aluminum, beryllium, barium, calcium, magnesium, sodium, potassium, iron, zirconium, titanium and a mixture of at least two thereof. The metal oxide is preferably selected from the group consisting of aluminum oxide (Al₂O₃), magnesium oxide (MgO), zirconium oxide (ZrO₂), yttrium oxide (Y₂O₃), aluminum titanate (Al₂TiO₅), a piezoceramic such as lead zirconate (PbZrO₃), lead titanate (PbTiO₃) or lead zirconate titanate (PZT) and a mixture of at least two thereof. The semimetal of the semimetal oxide is preferably selected from the group consisting of boron, silicon, arsenic, tellurium and a mixture of at least two thereof.

The silicate ceramic is preferably selected from the group consisting of a steatite (Mg₃[Si₄O₁₀(OH)₂]), cordierite ((Mg, Fe²⁺)₂(Al₂Si)[Al₂Si₄O₁₈]), mullite (Al₂Al₂₊₂ₓSi₂₋₂ₓO₁₀₋ₓ where x=oxygen vacancies per unit cell), feldspar ((Ba,Ca,Na,K,NH₄)(Al,B,S)₄O₈) and a mixture of at least two thereof.

The nonoxidic ceramic can be selected from the group consisting of a carbide, a nitride and a mixture thereof. The carbide can be selected from the group consisting of silicon carbide (SiC), boron carbide (B₄C), titanium carbide (TiC), tungsten carbide, cementite (Fe3C). The nitride can be selected from the group consisting of silicon nitride (Si₃N₄), aluminum nitride (AlN), titanium nitride (TiN), silicon aluminum oxynitride (SIALON) and a mixture of at least two thereof.

For the purposes of one embodiment of the invention, a “cermet” is a composite composed of one or more ceramic materials in at least one metallic matrix or a composite composed of one or more metallic materials in at least one ceramic matrix. To produce a cermet, it is possible to use, for example, a mixture of at least one ceramic powder and at least one metallic powder which can be, for example, admixed with at least one binder and optionally at least one solvent. A selection of the ceramic constituents and the metallic constituents of the cermet can be made up of those indicated for the first hollow body.

The at least one further hollow body or at least one further hollow body precursor thereof preferably comprises the additive in an amount in the range from 0.1 to 5% by weight, or preferably in the range from 0.2 to 2% by weight, or preferably in the range from 0.3 to 1% by weight, in each case based on the total weight of the at least one further hollow body or the hollow body precursor.

The at least one first hollow body and the at least one further hollow body can be arranged in different ways within the pump housing. In a preferred embodiment of the pump housing, at least one surface of the at least one further hollow body faces the outside of the pump housing and at least one surface of the at least one first hollow body faces the interior region. This will also be referred to as the side facing away from the interior region. The at least one first hollow body or the at least one further hollow body can form the entire wall thickness in a cross section of the pump housing at one point at least in part of the pump housing. The wall of the pump housing is then formed at these places on the cross section only by a single hollow body, i.e. the first hollow body or the second hollow body. As an alternative, the wall of the pump housing can have at least one first hollow body and at least one further hollow body in a cross section through a point on the pump housing. The first hollow body preferably faces the side of the interior region of the pump housing, while the further hollow body preferably faces the outside of the pump housing. This can be achieved, for example, by insertion of two tubes into one another, with the first tube representing the first hollow body and the second tube representing the further hollow body.

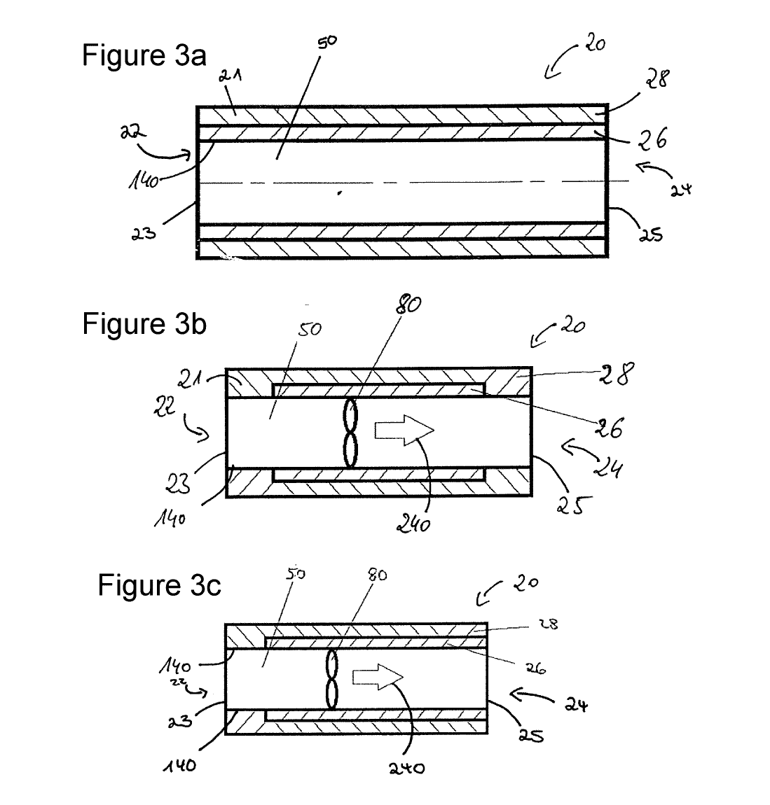

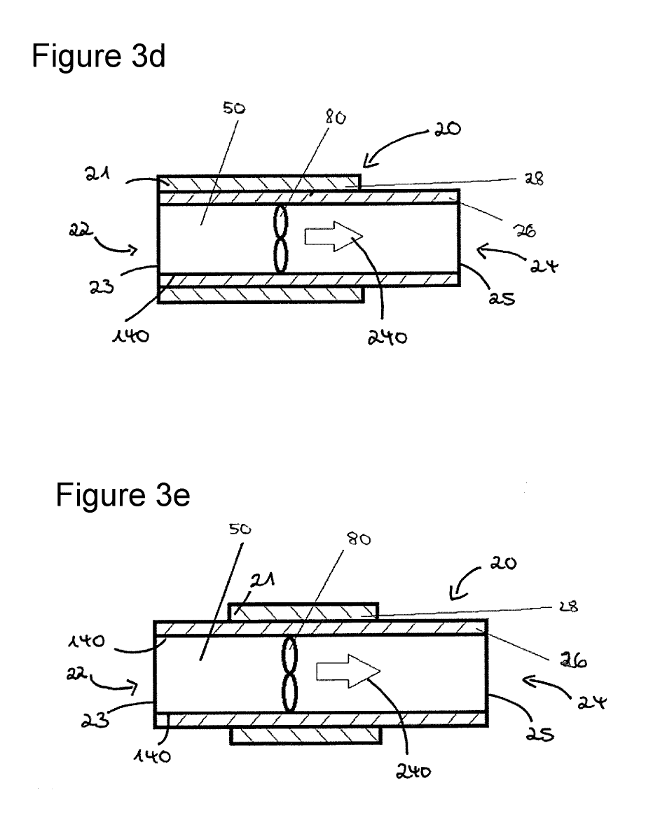

Each transition from one hollow body to another hollow body can be arranged at right angles or at an angle other than 90° relative to a cross section of the pump housing. Furthermore, each transition can also have an irregular configuration, i.e. viewed in cross section, no imaginary straight line can be drawn on the transition. Furthermore, each transition from one hollow body to another hollow body can, as an alternative to or in addition to what has been described above, be arranged at right angles or at an angle other than 90° relative to a longitudinal section through a wall of the pump housing. Furthermore, each transition can also have an irregular configuration, i.e. viewed in longitudinal section, no imaginary straight line can be drawn on the transition. Furthermore, combinations of the abovementioned configurations of a transition in cross section and in longitudinal section are preferred. Examples are shown in FIGS. 3a to 3e.

The pump housing preferably has various sections in which the pump housing comprises only the first hollow body or only the further hollow body or both hollow bodies along its longitudinal extension. These sections can alternate over the total length of the pump housing.

In a preferred embodiment of the pump housing of the pump device of one embodiment of the invention, both the first hollow body and the further hollow body extend over the entire length of the pump housing. This is shown by way of example in FIG. 3a. In another embodiment of the pump housing of the pump device of one embodiment of the invention, the further hollow body extends over the entire length of the pump housing, but the first hollow body extends over only part of the length of the pump housing. This is shown by way of example in FIGS. 3b and 3c. Furthermore, in another embodiment of the pump housing of the pump device of one embodiment of the invention, the first hollow body extends over the entire length of the pump housing and the further hollow body extends over only part of the length of the pump housing. This is shown by way of example in FIGS. 3d and 3e. In a further embodiment of the pump housing of the pump device of one embodiment of the invention, the pump housing has only the first hollow body or the further hollow body at the inlet. This is shown by way of example in FIGS. 3b, 3c and 3e. A further preferred embodiment of the pump housing of the pump device of one embodiment of the invention has only the first hollow body or the further hollow body at the outlet of the pump housing. This is shown by way of example in FIGS. 3b, 3d and 3e.

Furthermore, in an embodiment of the pump housing of the pump device of one embodiment of the invention it is preferred if a part of the surface of the further hollow body faces the interior region. This is shown by way of example in FIGS. 3b and 3c. In a further preferred embodiment of the pump housing of the pump device of one embodiment of the invention, only the further surface of the first hollow body faces the interior region. This is shown by way of example in FIGS. 3a, 3d and 3e.

The pump device of one embodiment of the invention additionally comprises a rotor in the form of the impeller. The impeller can have any shape which a person skilled in the art would select for this purpose.

The impeller preferably has a diameter in the range from 1 mm to 10 cm, preferably in the range from 3 mm to 5 cm, or preferably in the range from 5 mm to 3 cm. The impeller preferably has a thickness in the range from 0.1 to 50 mm, preferably in the range from 0.5 to 20 mm, or preferably in the range from 1 to 15 mm. The diameter of the impeller is preferably smaller than the diameter of the pump housing in the plane of the impeller. The diameter of the impeller is preferably from 1 to 10% smaller, or preferably from 1.5 to 8% smaller, or preferably from 2 to 7% smaller than the diameter of the pump housing, based on the diameter of the pump housing in the plane of the impeller.

The impeller preferably has at least two rotor blades, preferably at least three rotor blades, or preferably at least five rotor blades. The impeller particularly preferably has a number of rotor blades in the range from 2 to 20, preferably in the range from 5 to 15, or preferably in the range from 8 to 13. The impeller preferably has a central axis of rotation about which the impeller can be rotated. The axis of rotation will also be referred to as rotational axis. The at least two rotor blades are preferably arranged symmetrically around the axis of rotation of the impeller. The impeller is preferably arranged in the interior region of the pump housing, with the rotational axis of the impeller being provided parallel to the longitudinal extension of the wall of the tube.

The impeller can be made of any material which a person skilled in the art would select for use in the pump device of one embodiment of the invention. The impeller preferably has at least two regions: a first region in the center of the impeller around the rotational axis—this first region will also be referred to as core region —, and a second region, also referred to as rotor region. This second region has at least two rotor blades which are suitable for conveying the fluid to be conveyed.

The impeller comprises at least one element which has hard-magnetic properties. A hard-magnetic property means that a material acquires permanent magnetization as a result of placing this material in a magnetic field. The strength of a magnetizing field is selected as a function of the composition of the element. The considerations and calculations required for this purpose will be well known to a person skilled in the art. When carrying out the magnetization, the induction of the impeller is preferably saturated. After the magnetic field has decreased, the magnetization of the hard-magnetic material remains. Materials having hard-magnetic properties can be used as permanent magnets. The at least one element is preferably arranged on the impeller in such a way that it moves the impeller when it is alternately attracted or repelled by two mutually independent electric or magnetic fields. The impeller preferably comprises at least two elements having hard-magnetic properties. Furthermore, the impeller can be controlled in respect of its radial or else axial alignment by means of at least one optional element. The elements having hard-magnetic properties are preferably utilized for mounting the impeller with as little contact as possible in the pump housing without further auxiliary means such as bearings or other fixings in the pump housing. This makes particularly low-friction and particularly low-wear operation possible.

The at least one element can, for example, be realized by means of at least one rotor blade which comprises a hard-magnetic material. As an alternative, a hard-magnetic element can be arranged on at least one rotor blade. The hard-magnetic element is preferably provided in the core of the impeller. The at least one hard-magnetic element preferably comprises at least one magnetizable material such as iron, cobalt, nickel, chromium dioxide or a mixture of at least two thereof. The at least one element can, for example, be arranged in the form of a coating composed of hard-magnetic material on at least one rotor blade or in the interior of the impeller. At least 50%, or preferably at least 70%, or preferably 100%, of the rotor blades preferably comprise a hard-magnetic material. The element preferably comprises at least 10% by weight, or preferably at least 20% by weight, or preferably at least 30% by weight, based on the total mass of the element, of a hard-magnetic metal. Furthermore, the element preferably comprises a cobalt-chromium alloy or a platinum-cobalt alloy, in particular a platinum-cobalt alloy (PtCo23) having a proportion of cobalt of 23% by weight, based on the total mass of the alloy, in the range from 10 to 100% by weight, or preferably in the range from 20 to 100% by weight, or preferably in the range from 30 to 100% by weight, based on the total mass of the element.

The impeller can have a different material in its core, i. e. the region around the axis of rotation, than in or on the rotor blades. As an alternative, the impeller can comprise a uniform material in the core and in the rotor blades. The material of the rotor blades can be flexible or inflexible. The material of the core of the impeller or the rotor blades of the impeller is in each case preferably selected from the group consisting of a polymer, a metal, a ceramic and a combination or mixture of at least two thereof.

The polymer can be selected from the group consisting of a chitosan, a fibrin, a collagen, a caprolactone, a lactide, a glycolide, a dioxanone, a polyurethane, a polyimide, a polyamide, a polyester, a polymethyl methacrylate, a polyacrylate, a Teflon, a copolymer of at least two thereof and a mixture of at least two thereof.

The metal can be selected from the group consisting of iron (Fe), stainless steel, platinum (Pt), iridium (Ir), niobium (Nb), molybdenum (Mo), tungsten (W), titanium (Ti), cobalt (Co), chromium (Cr), a cobalt-chromium alloy, tantalum (Ta), vanadium (V) and zirconium (Zr) and a mixture of at least two thereof, with particular preference being given to titanium, niobium, molybdenum, cobalt, chromium, tantalum, zirconium, vanadium and alloys thereof.

The ceramic can be selected from the group consisting of aluminum oxide (Al₂O₃), zirconium dioxide (ZrO₂), hydroxyapatite, tricalcium phosphate, glass-ceramic, aluminum oxide-reinforced zirconium oxide (ZTA), zirconium oxide-containing aluminum oxide (ZTA—Zirconia Toughened Aluminum—Al₂O₃/ZrO₂), yttrium-containing zirconium oxide (Y-TZP), aluminum nitride (AlN), magnesium oxide (MgO), piezoceramic, barium (Zr, Ti) oxide, barium (Ce, Ti) oxide and sodium potassium niobate and a mixture of at least two thereof.

It is furthermore preferred that the impeller is coated on its outside, in particular on the outer surface of the rotor blades, with a biocompatible material. Suitable biocompatible materials are described below.

In the pump housing, the impeller is preferably arranged in the first hollow body of the pump housing and is thus surrounded by the first hollow body. The impeller is preferably arranged with its rotational axis parallel to the longitudinal extension of the wall. Furthermore, the impeller can be aligned in the pump housing by means of a magnetic field. The impeller in the interior region of the pump housing is preferably aligned by means of magnetic fields of electric coils on the outside of the pump housing. The coils preferably comprise an electrically conductive material. The electrically conductive material of the coils is preferably selected from the group consisting of iron (Fe), copper (Cu), gold (Au), silver (Ag), platinum (Pt), palladium (Pd), titanium (Ti), chromium (Cr), cobalt (Co), tungsten (W) and a mixture of at least two thereof. The electrically conductive material further preferably comprises copper (Cu). The pump device of one embodiment of the invention preferably comprises at least two coils, preferably at least three coils, or preferably at least four coils. The coils are preferably arranged on the outside of the pump housing, with the coils and the impeller preferably lying in one plane. They are then arranged on the outside of the pump housing around the impeller.

In a preferred embodiment of the pump device of one embodiment of the invention, the first hollow body and the further hollow body are connected in a force-fitting manner.

In a preferred embodiment of the pump device of one embodiment of the invention, the first hollow body or the further hollow body forms a cylindrical hollow body. The first hollow body and the further hollow body preferably each form a cylindrical hollow body.

In a preferred embodiment of the pump device of one embodiment of the invention, the pump housing comprises a tube. The tube is preferably straight. As an alternative, the tube can have at least one bend. The tube is preferably closed except for an inlet and an outlet. This means that the tube has no further openings apart from the two openings at the inlet and the outlet. The dimensions, materials and configurations preferably otherwise correspond to those of the above-described pump housing.

In a preferred embodiment of the pump device of one embodiment of the invention, the material of the first hollow body is a ceramic which is preferably selected from the group consisting of aluminum oxide (Al₂O₃), zirconium dioxide (ZrO₂), aluminum oxide-reinforced zirconium oxide (ZTA), zirconium oxide-reinforced aluminum oxide [(ZTA—Zirconia Toughened Aluminum—Al₂O₃/ZrO₂)], yttrium-reinforced zirconium oxide (Y-TZP), aluminum nitride (AlN), magnesium oxide (MgO), piezoceramic, barium (Zr, Ti) oxide, barium (Ce, Ti) oxide, titanium nitride (TiN), silicon dioxide (SiO₂) and sodium potassium niobate and a mixture of at least two thereof.

The at least one further hollow body of the pump housing has a metal content in the range from 50 to 90% by weight, preferably in the range from 55 to 85% by weight, or preferably in the range from 60 to 80% by weight, based on the total mass of the further hollow body.

In a preferred embodiment of the pump device of one embodiment of the invention, the metal of the further hollow body is selected from the group consisting of platinum (Pt), iron (Fe), stainless steel, in particular AISI 304 or AISI 316 L, iridium (Ir), niobium (Nb) a niobium alloy, molybdenum (Mo), tungsten (W), titanium (Ti), a titanium alloy, cobalt (Co), chromium (Cr), a cobalt-chromium alloy, tantalum (Ta) and zirconium (Zr) and a mixture of at least two thereof. The metal is preferably selected from the group consisting of titanium, niobium, molybdenum, cobalt, chromium, tantalum and alloys thereof and a mixture of at least two thereof.

Furthermore, the at least one further hollow body can comprise further materials. The further material of the further hollow body can, as described above for the first hollow body, be selected from the group consisting of an additive, a ceramic, a cermet and a mixture thereof. The at least one further hollow body preferably comprises the further material in a proportion of from 10 to 650% by weight, or preferably in a proportion of from 11 to 545% by weight, or preferably in a proportion of from 13 to 40% by weight.

The ceramic of the further hollow body can be any ceramic which a person skilled in the art would choose for a pump device. The ceramic is preferably selected from the group consisting of an oxide ceramic, a silicate ceramic, a nonoxidic ceramic and a mixture of at least two thereof. The ceramic of the at least one further hollow body can be selected from the same group as are the ceramics indicated for the first hollow body. The at least one further hollow body preferably comprises the same ceramic as the at least one first hollow body. The further hollow body preferably comprises the ceramic in a proportion of from 1 to 50% by weight, or preferably in a proportion of from 5 to 45% by weight, or preferably in a proportion of from 10 to 40% by weight, based on the total mass of the further hollow body.

Furthermore, the at least one further hollow body can comprise an additive as has been described for the first hollow body. The additive preferably comprises a material selected from the group consisting of water, a dispersant, a binder and a mixture of at least two thereof. All further properties and amounts of the additives can be taken from what has been said in respect of the first hollow body. The sum of all constituents of the further hollow body is always 100% by weight.

A selection of the ceramic constituents and the metallic constituents of the cermet can be made up of those indicated for the at least one further hollow body.

The pump housing preferably comprises at least one first hollow body and at least one further hollow body. The pump housing can have a plurality of first hollow bodies and a plurality of further hollow bodies. The pump housing preferably has one first hollow body and one further hollow body. The at least one first hollow body and the at least one further hollow body can have the same size or as an alternative have different sizes. The at least one first hollow body and the at least one further hollow body preferably extend for the most part together over the total thickness of the pump housing wall. The thicknesses of the first and further hollow bodies are preferably identical and correspond, depending on the arrangement of the hollow bodies relative to one another, either individually or together, to the wall thickness indicated above for the pump housing wall. The at least one first hollow body preferably has a width, based on the longitudinal extension of the pump housing, in the range from 1 to 100 mm, preferably in the range from 2 to 70 mm, or preferably in the range from 3 to 50 mm. The at least one further hollow body preferably has a width in the same ranges as the first hollow body. If the pump housing has more than one first hollow body and/or more than one further hollow body, the plurality of first hollow bodies and/or the plurality of further hollow bodies preferably have identical widths. As an alternative, first hollow bodies having different widths and further hollow bodies having different widths can alternate.

Furthermore, the at least one first hollow body can comprise a metal. The metal can be any metal which a person skilled in the art would select for manufacturing the pump housing.

In a preferred embodiment of the pump device of one embodiment of the invention, the at least one first hollow body comprises less than 10% by weight, preferably less than 5% by weight, or preferably less than 3% by weight, based on the total mass of the first hollow body, of metal. The sum of all constituents of the first hollow body is always 100% by weight.

The metal of the first hollow body is preferably selected from the group consisting of platinum (Pt), iron (Fe), stainless steel (AISI 304, AISI 316 L), iridium (Ir), niobium (Nb), molybdenum (Mo), tungsten (W), titanium (Ti), cobalt (Co), chromium (Cr), a cobalt-chromium alloy, tantalum (Ta) and zirconium (Zr) and a mixture of at least two thereof. The metal is preferably selected from the group consisting of titanium, niobium, molybdenum, cobalt, chromium, tantalum and alloys thereof and a mixture of at least two thereof. The first hollow body preferably comprises the same metal as the further hollow body.

In a preferred embodiment of the pump device, the pump device is surrounded at least partly by a component housing, wherein at least part of the at least one further hollow body of the pump device is connected to the component housing. The connection of the component housing to at least part of the further hollow body of the pump housing preferably leads to a closed space between the component housing and the pump housing. The interior of the component housing of the pump device is preferably hermetically sealed from the environment. The medically implantable pump device proposed here according to one embodiment of the invention can be used, in particular, in a body of a human or animal user, in particular a patient. An implanted pump device is generally exposed to a fluid of a body tissue of the body. It is therefore generally important that neither does body fluid penetrate into the medical implantable apparatus nor do liquids exit from the medically implantable apparatus. To ensure this, the component housing of the medically implantable apparatus, and thus also the component housing and the pump housing of the pump device of one embodiment of the invention, should have very complete impermeability, in particular in respect of body fluids.

The pump device of one embodiment of the invention, in particular the connections of component housing to pump housing, are preferably hermetically sealed. Thus, the interior space of the pump housing is hermetically sealed from the exterior space. For the purposes of one embodiment of the invention, the term “hermetically sealed” means that, during intended use, no moisture and/or gases can penetrate through the hermetically sealed join over a customary period of 5 years. A physical parameter for determining the freedom from leaks of a connection or a component is the leakage rate. Freedom from leaks can be determined by means of leakage tests. Leakage tests are carried out using helium leakage testers and/or mass spectrometers and are specified in the standard Mil-STD-883G method 1014. The maximum permissible helium leakage rate is set down as a function of the internal volume of the apparatus to be tested. According to the methods specified in paragraph 3.1 of MIL-STD-883G, method 1014, and taking into account the volumes and cavities of the apparatuses to be tested when the present invention is employed, the maximum permissible helium leakage rate for the pump housings of one embodiment of the invention is 10⁻⁷ atm*cm³/sec or less. This means that the apparatus to be tested (for example the component housing and/or the pump device of one embodiment of the invention or the component housing with the connected pump housing) has a helium leakage rate of less than 1×10⁻⁷ atm*cm³/sec or less. In a particularly advantageous embodiment, the helium leakage rate is less than 1×10⁻⁸ atm*cm³/sec, in particular less than 1×10⁻⁹ atm*cm³/sec. For the purpose of standardization, the helium leakage rates mentioned can also be converted into the equivalent standard air leak rate. The definition of the equivalent standard air leak rate and the conversion calculation are given in the standard ISO 3530.

The pump device of one embodiment of the invention preferably comprises not only the impeller, the pump housing with a first hollow body and a further hollow body but preferably also a component housing in which further components of the pump device can be present. The further components of the pump device are preferably selected from the group consisting of a battery, a coil, a control unit, a vessel connection unit and a combination of at least two thereof.

In a preferred embodiment of the pump device of one embodiment of the invention, the pump housing has a volume in the range from 0.1 cm³ to 10 cm³, preferably in the range from 0.2 to 9 cm³, or preferably in the range from 0.5 to 5 cm³. The dimensions such as length, diameter and wall thickness of the pump housing are preferably as indicated above. The volume of the pump housing is preferably defined by its interior space and the wall thickness of the pump housing. The wall of the pump housing preferably has a thickness in the range from 0.1 to 5 mm, preferably in the range from 0.3 to 4 mm, and preferably in the range from 0.4 to 3 mm. The volume of the interior region can be calculated appropriately from the length and the internal diameter of the pump housing. The preferred ranges for the interior volume are likewise obtained correspondingly. In this context, the term wall thickness will be employed in the following. The wall thicknesses can vary on the interior surface of the pump housing in at least one of the first or further hollow bodies. Increasing the wall thickness at least one point on the pump housing can serve to hold the impeller in position, in at least one direction, in the pump housing.

In a preferred embodiment of the pump device of one embodiment of the invention, the component housing comprises titanium in a proportion of at least 30% by weight, preferably at least 50% by weight, or preferably at least 80% by weight, in each case based on the total mass of the component housing. Furthermore, the component housing preferably comprises titanium in a proportion of at least 99% by weight, based on the total mass of the component housing. Furthermore, the component housing can preferably comprise at least one other metal. The other metal can be selected from the same group as the metal of the further hollow body. The other metal is preferably selected from the group consisting of Fe, Al, V, Sn, Co, Cr, CoCr, Nb, stainless steel, Mb, TiNb and a mixture of at least two thereof. The component housing can preferably comprise the further metal in a proportion of from 1 to 70% by weight, or preferably in a proportion of from 5 to 50% by weight, or preferably in a proportion of from 10 to 20% by weight. The sum of all constituents of the component housing is always 100% by weight. Suitable titanium grades are given in ASTM B265-05, year 2011, for example grade 1 to 6.

In a preferred embodiment of the pump device of one embodiment of the invention, the wall of the pump housing has a magnetic permeability of less than 2μ, preferably less than 1.9μ, or preferably less than 1.8μ. The magnetic permeability is determined in accordance with the standard ASTM 773:2009, variant 01. The measurement time is 40 seconds.

In a preferred embodiment of the pump device of one embodiment of the invention, the further surface of the first hollow body which faces the interior region of the pump housing has a Vickers hardness of at least 330 HV, preferably at least 350 HV, or preferably at least 370 HV. Preferably the entire at least one first hollow body has a hardness in the range as indicated. At least the outer surface of the at least one further hollow body likewise has a Vickers hardness of at least 330 HV, preferably at least 350 HV, or preferably at least 370 HV. The hardness is often not greater than 2000 HV, or preferably not greater than 1500 HV. The hardness at least of the surface of the at least one first hollow body is preferably in the range from 330 to 2000 HV, or preferably in the range from 350 to 1800 HV. Furthermore, at least the further surface of the at least one first hollow body preferably has a hardness which is at least as great as the hardness of the rotor surfaces of the impeller. At least the surface of the at least one first hollow body preferably has a hardness which is at least 20 HV greater, or preferably at least 30 HV greater, or preferably at least 40 HV greater, than the Vickers hardness of the rotor surfaces of the impeller. The surface of the at least one hollow body, of the at least one further hollow body and of the impeller is the layer of material close to the surface in a region of from 0.01 to 2.5 mm, preferably in a region of from 0.05 to 1.5 mm, or preferably in a region of from 0.1 to 1 mm, in each case perpendicular to the surface.

In a preferred embodiment of the pump device of one embodiment of the invention, at least the outer surfaces of the component housing and the surface facing the interior region of the pump housing are biocompatible. This is particularly preferred when the pump device is destined for implantation in a human body, for example that of a human being or animal. The biocompatibility is determined and assessed in accordance with the standard ISO 10993-4:2002.

In general, the surfaces facing the interior region of the pump housing and the outer surfaces of the component housing come into contact with body fluid after implantation of the pump device in a living body. The biocompatibility of the surfaces which come into contact with body fluid contributes to the body not being damaged on contact with these areas.

One aspect of the present invention further provides a process for producing a pump housing for a pump device, which comprises the steps:

a. Provision of a first hollow body;

b. Provision of a further hollow body;

wherein at least the first hollow body is configured as a cylindrical hollow body;

c. Contacting of the first hollow body with the further hollow body to form a composite,

wherein the contacting is performed as a force-fitting or material-fitting connection of the first hollow body and the further hollow body.

The provision of the first hollow body in step a. and of the further hollow body in step b. can be carried out in any way which a person skilled in the art would select for this purpose.

The contacting in step c. can be any contacting which a person skilled in the art would use for a force-fitting or material-fitting connection between the first hollow body and the further hollow body.

In a preferred embodiment of the process for producing a pump housing according to one embodiment of the invention, the contacting in step c. is selected from the group consisting of adhesive bonding, pressing, sintering and a combination of at least two thereof. The contacting is preferably selected from the group consisting of pressing with hot contacting, sintering and adhesive bonding and a combination of at least two thereof. According to one embodiment of the invention, a composite of the first hollow body and the further hollow body is produced in step c. This composite of the two hollow bodies forms a stable pump housing. The bond between the two hollow bodies is preferably so strong that an attempt to separate the two hollow bodies would lead to destruction of the pump housing.

Hot contacting is preferably carried out by means of heating of one of the two hollow bodies. The further hollow body is preferably heated to a temperature which is below the melting point of the further hollow body but results in expansion of the hollow body. The further hollow body likewise preferably has a cylindrical shape. Furthermore, the further hollow body preferably has an internal diameter which is identical to the external diameter of the first hollow body. The hollow body is preferably brought to an elevated temperature, so that its internal diameter is somewhat increased and the further hollow body can be pushed over the first hollow body. The further hollow body is preferably heated to a temperature in the range from 200 to 400° C., or preferably in the range from 220 to 350° C., or preferably in the range from 240 to 300° C. On cooling of the further hollow body, the diameter thereof decreases and a frictional bond is formed between the first hollow body and the further hollow body. A material-fitting connection between two objects such as the first hollow body and the further hollow body can produce an equally strong bond, for example a material-fitting connection as is formed, for example, in the case of adhesive bonding or sintering.

Contacting by adhesive bonding can be carried out using any adhesive which a person skilled in the art would select for this purpose. The two hollow bodies preferably both have a cylindrical shape. The internal diameter of the further hollow body is preferably somewhat greater than the external diameter of the first hollow body. The further hollow body does not in this case have to have a closed longitudinal extension. The further hollow body can, for example, be a slitted elongated, hollow body.

The adhesive can be selected from the group consisting of polyurethane, polyacrylates, polyesters, polyvinyl alcohols, polysulfones and a mixture of at least two thereof. A solvent for the polymers is preferably selected from the group consisting of dimethyl sulfoxide (DMSO), ethylene glycol, N-methyl-2-pyrrolidone (NMP), ammonia, water, an alcohol such as ethanol, isopropanol or hexanol and a mixture of at least two thereof. Furthermore, a crosslinker, for example, can be used. The crosslinker can be, for example, a silane.

In a further embodiment of the process of one embodiment of the invention, the first hollow body can be in the form of a fired ceramic. The first hollow body is preferably in the form of a tube having a length of 2 cm with an internal diameter of 9 mm. At least one layer of a further material from which the further hollow body is formed is then preferably applied to the ceramic at at least one place on the first hollow body by screen printing. The screen printing operation can be repeated until a sufficient layer, for example in the range from 0.2 to 5 mm, or preferably in the range from 0.5 to 4.5 mm, or preferably in the range from 0.6 to 4.3 mm, of further material for the further hollow body has been applied on the end of the first hollow body. As an alternative, both hollow bodies can be produced in succession by screen printing. Contacting in the case of sintering is preferably carried out by contacting of two precursors of the hollow bodies. These can together also be referred to as pump housing precursors. For this purpose, a cylindrical precursor of a first hollow body is preferably provided, preferably as unfired composition of a ceramic material as described above for the first hollow body. An unfired material selected from among the constituents of the further hollow body as described above for the pump device is subsequently applied to the outer surface. The unfired material is preferably in the form of a paste or powder. The two at least partly surrounding first and further hollow bodies are together subjected to a sintering process. The sintering process preferably takes place at a temperature in the range from 500 to 2500° C., or preferably in the range from 700 to 2000° C., or preferably in the range from 1000 to 1700° C. The treatment of the hollow body precursor is carried out for a period of time in the range from 0.1 to 100 hours, preferably in the range from 1 to 50 hours, or preferably in the range from 2 to 20 hours.

Furthermore, the at least one first hollow body precursor and the at least one further hollow body precursor can comprise an additive. The additive can be selected from the group consisting of water, a dispersant, a binder and a mixture of at least two thereof.

The dispersant preferably comprises at least one organic substance. The organic substance preferably has at least one functional group. The functional group can be a hydrophobic functional group or a hydrophilic functional group. The functional group can be selected from the group consisting of an ammonium group, a carbon/late group, a sulfate group, a sulfonate group, an alcohol group, a polyalcohol group, an ether group and a mixture of at least two thereof. The dispersant preferably has from 1 to 100, or preferably from 2 to 50, or preferably from 2 to 30, functional groups. Preferred dispersants are obtainable under the trade names DISPERBYK® 60 from Byk-Chemie GmbH, DOLAPIX CE 64 from Zschimmer & Schwarz GmbH & Co KG.

The binder is preferably selected from the group consisting of a methylcellulose, a thermoplastic polymer, a thermoset polymer and a wax and a mixture of at least two thereof.

The methylcellulose is preferably selected from the group consisting of hydroxylpropylmethylcellulose (HPMC), hydroxyethylmethylcellulose (HEMC), ethylmethylcellulose (EMC) and a mixture thereof. The methylcellulose preferably comprises hydroxypropylmethylcellulose (HPMC). Further preferably, the methylcellulose comprises hydroxypropylmethylcellulose in a proportion of from 80 to 100% by weight, or preferably in a proportion of from 90 to 100% by weight, or preferably in a proportion of from 95 to 100% by weight, based on the total mass of methylcellulose. The methylcellulose preferably has a proportion of —OCH₃ groups in the range from 20 to 40% by weight, or preferably in the range from 23 to 37% by weight, or preferably in the range from 25 to 35% by weight, based on the total mass of methylcellulose. Furthermore, the methylcellulose preferably has a proportion of —OC₃H₆OH groups in the range from 1 to 12% by weight, or preferably in the range from 3 to 9% by weight, or preferably in the range from 4 to 8% by weight, based on the total mass of methylcellulose.

The thermoplastic polymer can be selected from the group consisting of acrylonitrile-butadiene-styrene (ABS), polyamides (PA), polylactate (PLA), polymethyl methacrylate (PMMA), polycarbonate (PC), polyethylene terephthalate (PET), polyethylene (PE), polypropylene (PP), polystyrene (PS), polyether ether ketone (PEEK) and polyvinyl chloride (PVC) and a mixture of at least two thereof. The thermoset polymer can be selected from the group consisting of an aminoplastic, an epoxy resin, a phenolic resin, a polyester resin and a mixture of at least two thereof. Waxes are hydrocarbon compounds which melt without decomposition above 40° C. These can include polyesters, paraffins, polyethylenes or copolymers of at least two thereof.

The material for forming the first hollow body or the further hollow body preferably comprises at least one of the abovementioned additives, preferably in a proportion of from 0.1 to 10% by weight, or preferably in a proportion of from 0.2 to 8% by weight, or preferably in a proportion of from 0.5 to 5% by weight, based on the total mass of the first material.

In an embodiment of the process, the first hollow body can be in the form of a fired ceramic. The first hollow body is preferably in the form of a tube having a length of 2 cm with an internal diameter of 9 mm. At least one layer of a further material from which the further hollow body is formed is then preferably applied by screen printing to the ceramic at at least one place on the first hollow body. The screen printing operation can be repeated until a sufficient layer, preferably in the range from 0.2 to 5 mm, or preferably in the range from 0.5 to 4.5 mm, or preferably in the range from 0.6 to 4.3 mm, of further material for the further hollow body has been applied to the end of the first hollow body. As an alternative, both hollow bodies can be produced in succession by screen printing. The contacting in the case of sintering is preferably carried out by contacting of two precursors of the hollow bodies. These can also be referred to together as pump housing precursors. For this purpose, a cylindrical precursor of a first hollow body is firstly preferably provided, preferably as unfired composition of a ceramic material as described above for the first hollow body. An unfired material selected from among the constituents of the further hollow body as described above for the pump apparatus is subsequently applied to the outer surface. The unfired material is preferably in the form of a paste or powder. The two at least partly surrounding first and further hollow bodies are subjected together to a sintering process. The sintering process preferably takes place at a temperature in the range from 500 to 2500° C., or preferably in the range from 700 to 2000° C., or preferably in the range from 1000 to 1700° C. The treatment of the hollow body precursor is carried out over a period of time in the range from 0.1 to 100 hours, preferably in the range from 1 to 50 hours, or preferably in the range from 2 to 20 hours. Furthermore, the at least one first hollow body precursor and the at least one further hollow body precursor can comprise an additive. The additive can be selected from the group consisting of water, a dispersant, a binder and a mixture of at least two thereof.

Furthermore, machining can be combined with any other shaping process, in particular the contacting in step c. Machining involves structuring a solid body through use of machining tools, such as a drill or a punch. During structuring, part of the material is removed. In this way, solid bodies can be converted, for example, into hollow bodies. For example, a hollow space can be formed in the pump housing precursor by machining when the pump housing precursor is configured as a solid body. However, machining can also be a treatment step after production of a pump housing or housing. In addition to machining, polishing can also take place after the production of the pump housing.

At least part of the binder escapes during the sintering treatment of the pump housing precursor at elevated temperature. Various temperature profiles are possible during the treatment in step c. of the pump housing precursor. The treatment of the pump housing precursor can, for example, be carried out in an oxidative atmosphere, a reductive atmosphere or under a protective atmosphere. An oxidative atmosphere can, for example, contain oxygen, e.g. air or an oxygen/air mixture. A reductive atmosphere can, for example, contain hydrogen. A protective atmosphere preferably comprises neither oxygen nor hydrogen. Examples of protective atmospheres are nitrogen, helium, argon, krypton and mixtures thereof. The choice of the atmosphere can be dependent on the materials to be treated. A person skilled in the art will be familiar with the suitable choice of the atmosphere for the materials mentioned. It can also be preferred for combinations of different atmospheres to be selected in succession for various periods of time.

The treatment of the pump housing precursor can be carried out either in one step or preferably in more than one more step. The pump housing precursor is preferably treated, in a first substep, at a temperature in the range from 301 to 600° C., or preferably in the range from 350 to 550° C., or preferably in the range from 400 to 500° C. This first substep can be carried out over a period of time in the range from 0.1 to 100 hours, preferably in the range from 1 to 50 hours, or preferably in the range from 2 to 20 hours. This substep can be carried out either by introduction of the pump housing precursor from step c. into a preheated atmosphere or by slow stepwise or continuously increased heating of the pump housing precursor. The treatment in the first substep of the pump housing precursor is preferably carried out in one step at a temperature in the range from 301 to 600° C.

In a second substep of the treatment, which preferably follows the first substep, the pump housing precursor is preferably heated to a temperature in the range from 800 to 2500° C., or preferably in the range from 1000 to 2000° C., or preferably in the range from 1100 to 1800° C. This substep, too, can be carried out either by introduction of the pump housing precursor from the first substep into a preheated atmosphere or by slow stepwise or continuously increased heating of the pump housing precursor. The treatment in the second substep of the pump housing precursor is preferably carried out in one step at a temperature in the range from 800 to 2500° C. The treatment of the pump housing precursor in the second substep is carried out over a period of time in the range from 1 to 180 minutes, preferably in the range from 10 to 120 minutes, or preferably in the range from 20 to 100 minutes.

The shape of the pump housing after the production process is preferably continuous. This means that the pump housing has no further openings or outlets or other cut-outs apart from the outlet and the inlet. The pump housing preferably has a straight outer surface. The wall thicknesses on the interior surface of the pump housing can vary in at least one of the first or further hollow bodies. An increase in the wall thickness at at least one point on the pump housing can serve to hold the impeller in position, in at least one direction, in the pump housing. The thickening of the wall thickness can take place either during the production process or subsequent thereto. In addition or as an alternative thereto, the pump housing can have constrictions.

A pump device according to one embodiment of the invention is obtainable by insertion of an impeller into a pump housing, arrangement of electromagnets with coils around the pump housing, establishment of an electric circuit with inclusion of a control device and a power source, e.g. a battery. Preference is given to the pump device of one embodiment of the invention being surrounded by a component housing and the at least one further hollow body of the pump housing being connected in a material-fitting manner to the component housing. This can be effected, for example, by means of a soldered connection along the point of contact of pump housing and component housing.

One aspect of the present invention further provides a pump housing for a pump device obtainable by the above-described process of the invention.

One aspect of the present invention further provides a housing which at least partly surrounds an interior region and has a first end and a second end,

wherein the housing comprises a composite of a first hollow body and a further hollow body which at least partly surrounds the first hollow body on the side facing away from the interior region;

wherein at least 60% of the surface area of the side of the first hollow body facing away from the interior space is connected to the further hollow body, based on the surface area of the side of the first hollow body facing away from the interior space;

wherein the housing faces the interior region with a further surface of the first hollow body;

wherein the further surface of the first hollow body facing the interior region of the housing has a hardness of at least 330 HV,

wherein the further hollow body has a metal content of at least 50% by weight, based on the total mass of the further hollow body.

The housing corresponds in terms of its shape, its composition and its further configuration to the pump housing which has been described above in connection with the pump device.

In a preferred embodiment of the housing, a shiftable element is provided in the component housing at least in part of the component housing. Further preferred embodiments correspond to the above-described embodiments of the pump device.

The shiftable element can be selected from the group consisting of a sphere, a cylinder, an air bubble and a combination of at least two thereof. The shiftable element preferably has a shape which corresponds to the diameter of the pump housing. The material of the shiftable element can be any material which a person skilled in the art would use for this purpose. The shiftable element preferably comprises a metal, a polymer, a ceramic or a mixture thereof. The metal or the polymer can be selected from among a metal, a polymer and a ceramic as have been described for the first hollow body for the pump housing. The shiftable element can, for example, serve to be shifted in terms of its position in the component housing by means of a change in the fluid stream in the component housing. The change in the position can, for example, be detected by application of a magnetic field over the component housing.

One aspect of the present invention further provides a pump device comprising at least one component housing as described above or a pump housing obtainable by a process as described above.

### Measurement Methods

1. Determination of the Vickers hardness (HV):

The testing forces and materials were determined in accordance with the standard DIN EN ISO 6507-March 2006. The following testing forces and durations of action were used: 1 kg, 15 seconds. The testing temperature was 23° C.±1° C.

2. Determination of the magnetic permeability: The magnetic permeability was determined in accordance with the standard ASTM 773, 2009, variant 01.

3. Determination of the biocompatibility:

The biocompatibility is determined in accordance with the standard ISO 10993-4:2002.

4. Determination of the hermetic connection:

Leakage tests are carried out using helium leakage testers and/or mass spectrometers. A standard measurement method is specified in the standard Mil-STD-883G method 1014. The maximum permissible helium leakage rate is set down as a function of the internal volume of the apparatus to be tested. According to the methods specified in paragraph 3.1 of MIL-STD-883G, method 1014, and taking into account the volumes and cavities of the apparatuses to be tested occurring when the present invention is employed, the maximum permissible helium leakage rate for the pump housing of the invention is 10⁻⁷ atm*cm³/sec or less. This means that the apparatus to be tested (for example the component housing and/or the pump device or the component housing with the connected pump housing) has a helium leakage rate of less than 1×10⁻⁷ atm*cm³/sec or less. For comparative purposes, the abovementioned helium leakage rates can also be converted into the equivalent standard air leak rate. The definition of the equivalent standard air leak rate and the conversion calculation are given in the standard ISO 3530.

5. Determination of the roughness: DIN EN ISO 4288. Further parameter data: Maximum probe tip radius=2 μm; measurement distance=1.25 mm; wavelength limit=250 μm.

### Examples

### Example 1 for First Material

The first material contains 20 parts of aluminum oxide (Al₂O₃) obtainable from CeramTech GmbH, having a particle size of D₉₀=2 μm and a proportion of the binder METAWAX P-50 obtainable from Zschimmer & Schwarz GmbH & Co. KG.

### Example 2 for Further Material

The further material contains a mixture of 57% by weight of a platinum powder from Heraeus Precious Metals GmbH & Co. KG having a particle size D₅₀=50 μm, and 38% by weight of aluminum oxide (Al₂O₃) from CeramTech GmbH having a particle size of D₉₀=2 μm and 5% by weight of a binder METAWAX P-50 obtainable from Zschimmer & Schwarz GmbH & Co. KG.

### Example 3 for First Hollow Bodies

The first hollow body contains 100% by weight of aluminum oxide (Al₂O₃) from CeramTech GmbH.

### Example 4 for Further Hollow Bodies

The further hollow body contains 60% by weight of platinum and 40% by weight of aluminum oxide (Al₂O₃) from CeramTech GmbH.

The first material as per example 1 is firstly processed to form a first hollow body precursor. The further material as per example 2 is firstly processed to form a further hollow body precursor which at least partly surrounds the first hollow body precursor. A pump housing precursor according to one embodiment of the invention is obtained in this way. Heating of the pump housing precursor in the range from 1400 to 1800° C. gives a pump housing according to one embodiment of the invention which contains the first hollow body having the composition as per example 3 and a further hollow body having a composition corresponding to that of example 4.

As an alternative, the two hollow body precursors can be sintered separately and subsequently connected to one another in a force-fitting or material-fitting manner.

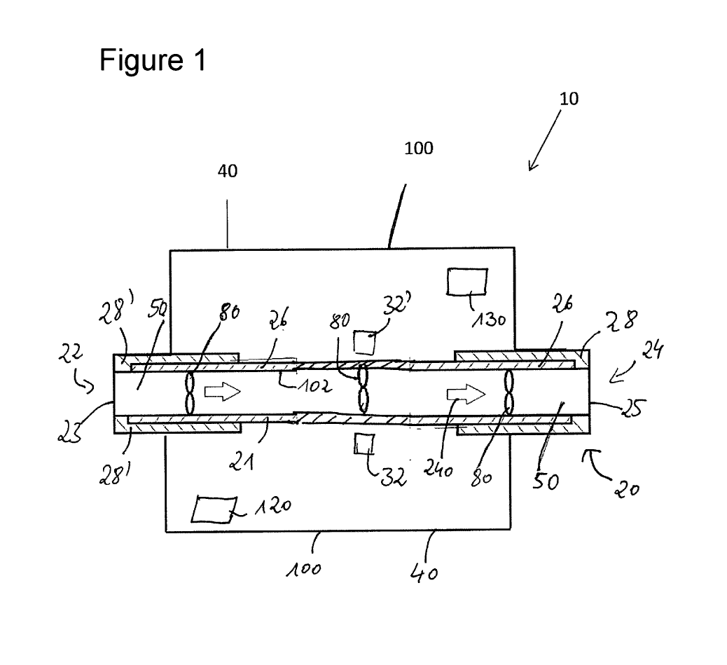

FIG. 1 schematically shows a pump device 10 which has a pump housing 20 in the form of a tube and also a component housing 40. The outer surfaces 100 of the component housing 40 come, particularly in the case of an implantable pump device 10, into contact with the body and are therefore preferably made biocompatible. The pump housing 20 has a wall 21 which surrounds an interior region 50. The surface of the pump housing 20 which faces the interior region 50 is referred to as facing surface 102. The facing surface 102 comes into contact with the fluid and is therefore preferably made biocompatible, especially for an implantable pump device 10. In the interior region 50 of the pump housing 20, there is at least one impeller 80; in this case, two impellers 80 are present in the pump housing 20. The pump housing 20 has a first hollow body 26 in the middle of the wall 21. At the first end 22, which at the same time defines the inlet 22 through the opening 23, the wall 21 or the pump housing 20 has a first further hollow body 28. On the opposite side of the pump housing 20, there is the further end 24, in the form of the outlet 24, comprising the further opening 25. A fluid can be pumped in the pumping direction 240 from the inlet 22 to the outlet 24 by means of the impeller 80. Further components such as a battery 120 and a control unit 130 are located between the component housing 40 and the pump housing. Furthermore, two coils 32 and 32′ are present in the component housing 40. In this example, the pump housing 20 of the pump device 10 has a first hollow body 26 and two further hollow bodies 28, 28′. The pump housing 20 can, as an alternative, also have only one first hollow body 26 and one further hollow body 28, as is shown in FIGS. 3a to 3e.

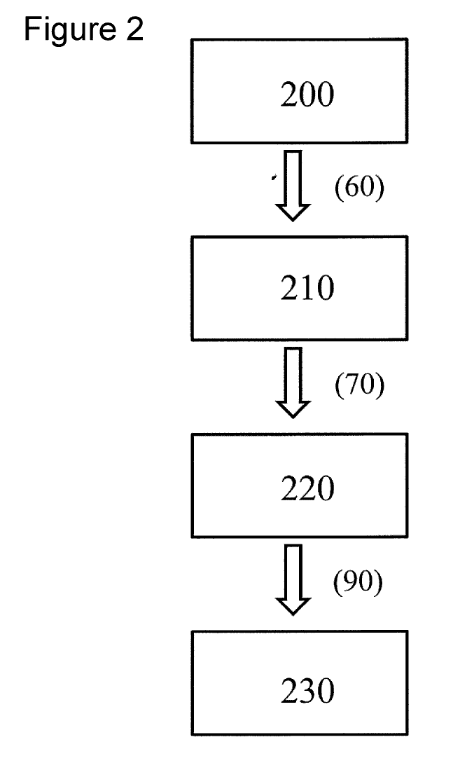

FIG. 2 shows a schematic flow diagram for the process for producing a pump housing. In step a. or a) 200, a first material 60 is provided. The first material 60 is, for example, a mixture of at least two powders. The first material contains the composition as per example 1, namely 20 parts of aluminum oxide (Al₂O₃) having a particle size of D₉₀=2 μm and one part of a binder, in this case METAWAX P-50 obtainable from Zschimmer & Schwarz GmbH & Co. KG.

The further material 70 is provided in the form of a mixture corresponding to example 2 of 57% by weight of a platinum powder having a particle size D₅₀=50 μm, and 38% by weight of aluminum oxide (Al₂O₃) having a particle size of D₉₀=2 μm and 5% by weight of a binder, in this case METAWAX P-50 obtainable from Zschimmer & Schwarz GmbH & Co. KG in a step b. or b) 210, likewise preferably in a vessel. The vessel can be a metal vessel having a screen outlet. The powder particles preferably have a round to oval shape. The particle size D₅₀ means that not more than 50% of the particles are larger than the diameter indicated. The particle size D₉₀ means that not more than 90% of the particles are larger than the diameter indicated. The particle size can be determined by various methods. The particle size is preferably determined by means of laser light scattering, optical microscopy, optical counting of individual particles or a combination of at least two thereof. Furthermore, the determination of the particle size is preferably carried out like the particle size distribution by means of optical individual analysis of transmission electron micrographs (TEM). In addition, the particle size can be taken from the product data sheet which is available from the raw material supplier and often accompanies a delivery.

The contacting in step c. can alternatively be carried out after shaping of the at least one first hollow body 26 or else before shaping of the at least one first hollow body 26. Shaping is sintering in this example. If contacting is, as in this example, carried out after sintering of the two hollow bodies 26 and 28, both a sintered hollow body 26 and a sintered hollow body 28 are contacted with one another in step c. In this example, contacting takes place by heating of the further hollow body 28 to a temperature of at least 250° C. in a convection oven from Heraeus Holding GmbH for 30 minutes and subsequently by pushing the two cylindrical hollow bodies 26 into the hollow body 28. The outer further hollow body 28 subsequently cools and shrinks in the process. In this way, the interior surface of the further hollow body 28 presses onto the outer surface of the first hollow body 26, so that a force-fitting connection between the two hollow bodies 26 and 28 is formed. This composite 142 represents, in the cooled state, the pump housing 20.

FIGS. 3a to 3e show various possible ways of arranging the first hollow body 26 and the further hollow body 28 in a pump housing 20 according to one embodiment of the invention. The wall 21 of the pump housing 20 has a first opening 23 at the inlet 22 and a further opening 25 at the outlet 24. In FIG. 3a, a first hollow body 26 is completely, i.e. from the inlet 22 to the outlet 24, surrounded by the further hollow body 28.

In FIG. 3b, the first hollow body 26 is, as a difference from the arrangement in FIG. 3a, also surrounded on the side of the inlet 22 and of the outlet 24 by the further hollow body 28. The interior surface of the pump housing 20 has both regions of the first hollow body 26 and regions of the further hollow body 28. The entire exterior area of the pump housing has the further hollow body 28. Furthermore, an impeller 80 which can pump a fluid in the pump direction 240 shown is arranged in the pump housing 20.

In FIG. 3c, the further hollow body 28 is, as a difference from the arrangement in FIG. 3b, pushed around the first hollow body only at the inlet 22. Here too, the entire outer surface area of the pump housing has the further hollow body 28.

FIG. 3d again has, like the arrangement of the pump housing 20 in FIG. 3a, the first hollow body 26 over the entire interior region 50. This means that the entire interior area 140 is formed by the further surface. On the outside of the pump housing 20, part of the first hollow body 26 is present at the outlet 24 and part of the further hollow body 28 is also present at the inlet 22. The inlet consequently comprises both the first hollow body 26 and the further hollow body 28.

FIG. 3e has only the first hollow body 26 at the inlet 22 and at the outlet 24. The further hollow body 28 is pushed over the first hollow body 26 only over the middle part of the pump housing 20. The interior area 140 of this pump housing 20 has only the first hollow body 26.

1-15. (canceled)

16. A pump device comprising:
a pump housing that at least partly surrounds an interior region and has an inlet and an outlet; and
an impeller provided in the interior region of the pump housing;
wherein the pump housing comprises a composite of a first hollow body and a further hollow body which at least partly surrounds the first hollow body on the side facing away from the interior region;
wherein at least 60% of the surface area of the side of the first hollow body facing away from the interior space is connected to the further hollow body, based on the surface area of the side of the first hollow body facing away from the interior space,
wherein a further surface of the first hollow body of the pump housing faces the interior region;
wherein the further surface of the first hollow body facing the interior region of the pump housing has a hardness of at least 330 HV,
wherein the further hollow body has a metal content of at least 50% by weight, based on the total mass of the further hollow body.

17. The pump device of claim 16, wherein the first hollow body and the further hollow body are connected in a force-fitting manner.

18. The pump device of claim 16, wherein the first hollow body or the further hollow body or both hollow bodies in each case form a cylindrical hollow body.

19. The pump device of claim 16, wherein the material of the first hollow body is a ceramic selected from the group consisting of aluminum oxide (Al₂O₃), zirconium dioxide (ZrO₂), aluminum oxide-reinforced zirconium oxide (ATZ), zirconium oxide-reinforced aluminum oxide (ZTA), yttrium-reinforced zirconium oxide (Y-TZP), aluminum nitride (AlN), magnesium oxide (MgO), piezoceramic, barium (Zr, Ti) oxide, barium (Ce, Ti) oxide, titanium nitride (TiN), silicon dioxide (SiO₂) and sodium potassium niobate and a mixture of at least two thereof.

20. The pump device of claim 16, wherein the metal of the further hollow body is selected from the group consisting of platinum (Pt), iron (Fe), stainless steel, iridium (Ir), niobium (Nb), a niobium alloy, molybdenum (Mo), tungsten (W), titanium (Ti), a titanium alloy, cobalt (Co), chromium (Cr), a cobalt-chromium alloy, tantalum (Ta) and zirconium (Zr) and a mixture of at least two thereof.

21. The pump device of claim 16, wherein the first hollow body comprises less than 10% by weight, based on the total mass of the first hollow body, of metal.

22. The pump device of claim 16, wherein the pump device is at least partly surrounded by a component housing, wherein the composite of the pump device is joined to the component housing.

23. The pump device of claim 16, wherein the pump housing has a volume in the range from 0.1 cm³ to 10 cm³.

24. The pump device of claim 16, wherein the component housing comprises at least 30% by weight, based on the total mass of the component housing, of titanium.

25. The pump device of claim 16, wherein the first hollow body has a magnetic permeability of less than 2μ.

26. A method for producing a pump housing for a pump device, the method comprising:
providing a first hollow body;
providing a further hollow body;
wherein at least the first hollow body is configured as a cylindrical hollow body;
contacting the first hollow body with the further hollow body to form a composite;
wherein the contacting is performed as a force-fitting or material-fitting connection of the first hollow body and the further hollow body.

27. The method of claim 26, wherein the contacting is selected from the group consisting of adhesive bonding, pressing, sintering and a combination of at least two thereof.

28. A pump housing obtainable by a process as claimed in claim 26.

29. A housing at least partly surrounding an interior region and having an inlet and an outlet, the housing comprising:
a composite of a first hollow body and a further hollow body at least partly surrounding the first hollow body on the side facing away from the interior region;
wherein at least 60% of the surface area of the side of the first hollow body facing away from the interior space is connected to the further hollow body, based on the surface area of the side of the first hollow body facing away from the interior space;
wherein a further surface of the first hollow body of the housing faces the interior region;
wherein the further surface of the first hollow body facing the interior region of the housing has a hardness of at least 330 HV; and
wherein the further hollow body has a metal content of at least 50% by weight, based on the total mass of the further hollow body.

30. A pump device comprising at least one housing as claimed in claim 29.
